(19) United States
(12) Patent Application Publication
(10) Pub. No.: US 2002/0129399 A1
(43) Pub. Date: Sep. 12, 2002

(54) Biotin-binding compounds for induction of sterility in plants

(76) Inventors: Marc Albertsen, Grimes, IA (US); Gary Huffman, Des Moines, IA (US)

Correspondence Address: Richard C. Peet, FOLEY & LARDNER, Washington Harbour, 3000 K Street, N.W., Suite 500, Washington, DC, 20007-5109, US

(21) Appl. No.: 09/801,928

(22) Filed: Mar. 9, 2001

(51) Int. Cl. A01H 1/02; C12N 15/82; C12N 15/29

(52) U.S. Cl. 800/274; 800/286; 800/287; 800/303; 536/23.6

(57) ABSTRACT

Sterile plants can be produced by increasing the endogenous concentration of a biotin-binding protein in the plant tissues, preferably those tissues that are critical to gamete formation or function. This effect can be achieved by producing transgenic plants containing an expression vector in which a promoter is operably linked to a DNA sequence encoding a biotin-binding polypeptide. Preferred biotin-binding proteins have a low susceptibility to degradation, are readily digested, or have a low allergenic potential. Other preferred polypeptides are not highly detrimental to cellular viability, such that low level expression in cells other than those critical for gamete formation or function may be tolerated more easily. This allows the use of promoters having low level expression in non-targeted tissues, thus expanding the repertoire of promoters useful for affecting sterility. Methods for restoring fertility are disclosed. Additionally, methods for production of seeds with one or more desired grain traits are provided.

### BACKGROUND OF THE INVENTION

Production of Hybrid Seed

The goal of plant breeding is to combine various desirable traits from the parental lines in a single variety or a hybrid. For field crops, these traits may include resistance to diseases and insects, better agronomic quality, tolerance to heat and drought, reduced time to crop maturity, and greater yield. Uniformity of plant characteristics, such as germination and stand establishment, growth rate, maturity, and fruit size, also is important for improving the efficiency of mechanical harvesting.

Field crops are bred through techniques that take advantage of the plant's method of pollination. A plant is self-pollinating if pollen from one flower is transferred to the same or another flower of the same plant. A plant is cross-pollinated if the pollen comes from a flower on a different plant. Brassica, for example, only cross-pollinates. In self-pollinating species, such as soybeans and cotton, the male and female plants are anatomically juxtaposed, such that the male reproductive organs of a given flower pollinate the female reproductive organs of the same flower. By contrast, maize plants (Zea mays L.) can self-pollinate and cross-pollinate. Maize has male flowers, located on the tassel, and female flowers, located on the ear, on the same plant. Natural pollination occurs when wind blows pollen from the tassels to the silks that protrude from the tops of the incipient ears.

The development of hybrids requires making homozygous inbred lines, crossing these lines, and evaluating the crosses. Breeding programs combine desirable traits from two or more inbred lines into breeding pools from which new inbred lines are developed by selfing and selection of desired phenotypes. Pedigree breeding and recurrent selection are two of the breeding methods used to develop inbred lines. A hybrid variety is the cross of two such inbred lines, each of which may have one or more desirable characteristics that are lacked by the other or that complement the other. The resulting hybrid is more vigorous than its inbred parents; i.e., it possesses hybrid vigor, as reflected by increased yield and vegetative growth, for example.

Use of Male-Sterile Plants in Hybrid Seed Production

A reliable method of controlling fertility in plants greatly facilitates plant breeding. For instance, the F₁ progeny of a completely male-sterile female inbred parent will be uncontaminated with progeny produced by self-pollination among the inbred line, and all the seed produced by the F₁ progeny accordingly are hybrid seed.

Genetic modification to produce male-sterile plants has proven advantageous over the more traditional methods of manual or mechanical detasseling or the use of cytoplasmic male-sterile lines. One type of genetic sterility is disclosed in U.S. Pat. Nos. 4,654,465 and 4,727,219 to Brar, et al. This method requires maintenance of multiple mutant genes at separate locations within the genome and requires a complex marker system to track the genes and make use of the system convenient. Patterson also describes a transgenic system of chromosomal translocations, which is effective but complicated. See U.S. Pat. Nos. 3,861,709 and 3,710,511.

Many improvements have been made to these methods. For example, Fabijanski et al. developed several methods of causing male sterility in plants. See EP 329,308 and WO 90/08828. In one method, a transgenic plant comprises a gene encoding a cytotoxic substance associated with a male tissue-selective promoter. In another method, an antisense gene reduces the expression of a gene that is critical to fertility. Mariani et al. and Cigan et al. describe operably linking cytotoxin-encoding gene sequences to male tissue-selective promoters to produce male-sterile plants. See EP 401,194 and U.S. Pat. No. 5,689,049, for example. Still other systems use “repressor” genes that inhibit the expression of a gene critical to male sterility. See WO 90/08829.

Use of Female-Sterile Plants in Hybrid Seed Production

The control of female fertility through genetic modification similarly offers advantages in plant breeding. After crossing with the male-sterile female inbred, the male inbred plant must be physically removed to prevent harvesting its seeds, which are produced by pollen from the male-fertile parent, thus adding to the expense of hybrid production. If the male inbred were female-sterile, however, the male and female parents could be harvested together, with considerable increase in efficiency.

Various methods have been used to control female fertility. DeGreef et al. describe a method of controlling female fertility in EP 412,006 and U.S. Pat. No. 5,633,441, which involves transforming plants with an expression vector that contains a female tissue-selective promoter linked to a dominant negative gene. The dominant negative gene encodes a product that disturbs the cellular development of the flower, seed, or embryo. For example, a barnase gene may be operably linked to an inducible promoter that directs expression selectively in style or stigma cells. Expression of the dominant negative gene is then induced by spraying the transgenic plant with a chemical that induces promoter activity. Albertsen et al. (U.S. Pat. No. 5,824,524) describe a contrasting system that involves transient induction of female fertility in an otherwise constitutively female-sterile transgenic plant. In this system, the inducible promoter is linked to a gene that restores the function of a gene that is critical to female gametogenesis.

Use of Avidin and Streptavidin to Affect Sterility

Transgenic control mechanisms that affect sterility typically require expression of a foreign protein that interferes with cell function, which is targeted to various cell populations that are crucial to the development of gametes. The biotin-binding proteins avidin and streptavidin have proven to be particularly useful proteins for this purpose. See U.S. Pat. No. 5,962,769, WO 96/40949, and WO 99/04023. It is believed that their effect on cellular function is related to their high affinity for biotin, which is an essential cofactor for a number of enzymes that are key to cellular viability. Its sequestration in the form of complexes with avidin or streptavidin would be expected to lead to cellular death as a result of the lack of activity of those enzymes that require biotin as a cofactor.

There is an ongoing need in the art to provide biotin-binding proteins capable of conferring male or female sterility when expressed in a plant. In particular, there is an ongoing need to provide an expanded repertoire of biotin-binding proteins with useful or improved properties, such as low susceptibility to degradation, good digestibility, or low allergenic potential.

There is a particular need to provide plant-derived genes encoding biotin-binding polypeptides. One advantage of plant-derived genes is that they will not require codon optimization for high level expression.

There also is a particular need to provide biotin-binding proteins that do not have a detrimental cellular effect when expressed at low levels. This advantageously would allow the use of tissue-selective promoters that are expressed at low levels in non-targeted tissue types. The activity of such promoters then could be further modulated by operably linking them with tissue-selective enhancer elements, for example. Thus, the repertoire of promoters useful for affecting sterility would be expanded greatly, since many tissue-selective promoters do not have the strict spatial and temporal regulation suitable for driving the expression of highly disruptive genes.

### SUMMARY OF THE INVENTION

These objectives are accomplished by providing a transgenic plant that expresses any of a variety of biotin-binding proteins. The transgenic plant comprises an expression vector containing one or more genes encoding the biotin-binding protein, which is operably linked to a promoter. In one embodiment of the invention, the promoter is expressed preferentially in cells that are critical to gamete formation or function. The transgenic plant that expresses the biotin-binding protein is thereby rendered either male- or female-sterile, and it is used advantageously in the preparation of hybrid seed.

Preferred biotin-binding proteins of the invention have low susceptibility to degradation or low allergenic potential. Among preferred biotin-binding proteins are those derived from plants. Thus, the biotin-binding proteins of the invention do not encompass avidin or streptavidin. Among preferred biotin-binding proteins are fragments or protein subunits, which are expressed more efficiently in transgenic organisms than the larger holoproteins. Representative biotin-binding polypeptides include SBP65, acetyl coenzyme A carboxylase, methylcronotyl-coenzyme A carboxylase, carbon-dioxide ligase, and pyruvate decarboxylase. A biotin-binding subunit of one of these enzymes or a biotin-binding fragment or derivative of one these enzymes may be encoded by the expression vector. The invention provides methods for preparing and modifying these genes and for constructing suitable expression vectors and transgenic plants.

To enhance the expression of the biotin-binding protein, the one or more genes encoding the biotin-binding protein may be linked to a regulatory sequence that activates promoter activity when a DNA binding protein is bound to the regulatory sequence. In one embodiment, the parent plant contains another gene that encodes a DNA binding protein that binds the regulatory sequence. A preferred DNA binding protein for this purpose is a fusion protein between LexA and C1. In another embodiment, a promoter is operably linked to one or more enhancer elements that are preferentially active in cells critical to gamete formation or function. The DNA-binding protein that binds the enhancer may be an endogenous plant protein, or it may be encoded by a foreign gene on an expression vector.

Male or female fertility may be restored simply by spraying the plant with a solution of biotin. Alternately, fertility is restored by crossing the sterile plant with a restorer line comprises a second gene, whose gene product reduces expression of the biotin-binding protein in the hybrid plant, thereby rendering it fertile. This second gene product may directly affect the stability of the mRNA of the biotin-binding gene. In this case, the second gene preferably is selected from the group consisting of an antisense gene, a ribozyme gene and an external guide sequence gene. Alternately, the restorer plant may encode a DNA-binding protein that binds to a regulatory element, which is operably linked to the promoter, to deactivate the promoter. For example, the second foreign gene may encode the LexA repressor, which deactivates promoter activity by binding to an operatively linked LexA binding site.

In a related embodiment, fertility may be restored by inducing the expression of a second foreign gene encoded by an expression vector in the sterile parent. In this embodiment, the gene encoding the biotin-binding protein and the second gene may be localized on the same expression vector used to make the transgenic plant. Expression of the second gene may be controlled by an inducible promoter, which may be induced by spraying a chemical inducer on the plant. As in the previous embodiment, the product of the second gene inhibits the expression of the biotin-binding protein to restore fertility. In this embodiment, the timing and location of restoration of fertility may be controlled through appropriate application of the inducer. Alternatively, the product of the second gene interferes with the biotin-binding ability of the biotin-binding protein.

The invention also provides a method for producing seeds having one or more grain or seed traits of interest, such as improved oil, protein, and starch content. In a preferred embodiment, a first male-sterile parent plant comprising a gene encoding a biotin-binding protein is crossed with a second parent plant that carries one or more grain or seed traits of interest. The first parent plant is either hemizygotic or homozygotic for the one or more copies of the gene encoding a biotin-binding protein.

A method of producing F₁ hybrid seeds is also provided. For this method, a first inbred parent plant is produced, which is male sterile and homozygotic for one or more copies of the gene encoding a biotin-binding protein. The first parent is crossed with a second inbred male-fertile parent. The male-sterile F₁ progeny is hemizygotic for the one or more copies of the gene encoding the biotin-binding protein. The F₁ plant is then crossed with a fourth parent plant which is male-fertile to produce the hybrid seeds. In one embodiment, the fourth parent plant carries one or more genes controlling a desired trait.

This method may be reiterated using these hybrid seeds to produce further parent plants possessing the one or more genes controlling the first desired gene trait. The fourth parent in the next iteration may possess one or more genes controlling yet another desired grain trait. Through these successive crosses, desired traits may be stacked into a single line.

Female fertility advantageously also may be manipulated during production of hybrid seed. In one embodiment, male inbred plants are made female-sterile by the expression of a biotin-binding protein. The male-fertile, female-sterile plant is harvested together with the male-sterile parent to produce hybrid seed solely from the male-sterile parent.

### BRIEF DESCRIPTION OF THE FIGURES

FIG. 1: Use of male sterility induced by biotin-binding proteins in the TopCross® production system.

FIG. 2: Use of male sterility induced by biotin-binding proteins to produce hybrid seed.

### DETAILED DESCRIPTION OF THE PREFERRED EMBODIMENTS

The present invention provides methods for generating transgenic female- or male-sterile plants by expressing a biotin-binding protein. The female- or male-sterile plant comprises at least one heritable gene encoding a biotin-binding polypeptide. The biotin-binding polypeptide interferes directly or indirectly with the development of a gamete, such that the transgenic plant is incapable of forming functional pollen or seeds.

A transgenic sterile plant embraces a plant that produces no gametes, a plant that produces only a small fraction of the gametes produced by the wild-type, or a plant that produces gametes that are substantially incapable of germination. A transgenic female-sterile plant further embraces a “functionally sterile” plant, in which pollen may germinate on the stigma, but the function of the stigma is impaired by the biotin-binding protein so that no seeds result from this pollination. In a preferred embodiment, no pollen at all is produced in male-sterile plants, and no seeds at all are produced by female-sterile plants. In a plant that produces a reduced amount of pollen, the proportion of seeds produced by self-pollination, even if low, may be commercially unacceptable. In a plant that produces non-functional pollen, manual detasseling may be required unless the seed producer can be assured that the pollen is totally nonfunctional prior to use of the male-sterile plant in hybrid seed production. These problems are avoided by the use of a male-sterile plant that produces no pollen.

The gene encoding a biotin-binding polypeptide is isolated and inserted into an expression vector suitable for introduction into plant tissue. An “expression vector” contains a promoter region, which is a DNA sequence that directs gene transcription. Typically, a promoter is located in the 5′ region of a gene, proximal to the transcriptional start site. Transcription from an “inducible promoter” is increased in response to an inducing agent. A “plant-compatible promoter” is a promoter sequence that will direct the transcription of a gene in plant tissue.

To make sterile plants, the biotin-binding protein preferably is expressed selectively in tissues critical for gamete formation or function. Selective expression in a tissue type means that a transcript accumulates to a relatively higher proportion of total mRNA in that tissue type compared to other tissue types. Selective expression may be conferred by using a promoter that is selectively active in the targeted tissue type. Alternately, or in addition, selective expression may be conferred by using enhancers that are selectively active in the targeted tissue type.

In the case of male-sterile plants, the expression of the biotin-binding protein is selective for tissues that are critical for pollen formation or function. In addition to pollen itself, preferred target tissues are devoted to the development or maintenance of pollen, including any tissue type found within the anther or stamen. For example, the biotin-binding protein may be expressed selectively in tapetum tissue or in micropores. Preferably, the gene is expressed in an anther-selective manner. Likewise, in the case of female-sterile plants, a female tissue-selective promoter is linked to the polynucleotide sequence encoding the biotin-binding protein. The biotin-binding protein disrupts cells critical to the development of seeds or embryos, which include the tissues of the style and stigma. For example, disruption of papillar cells or the secretory zone in the stigma may cause deposited pollen to fail to form seeds, even if the pollen germinates.

The expression vector may include a regulatory sequence. Regulatory sequences typically are located at the 5′ end of a gene and contain a nucleotide sequence that is recognized and bound by a repressor or activator protein. The binding of a repressor or activator protein with its cognate binding site results in transcriptional inhibition or activation. For example, the LexA repressor protein binds to the LexA operator to inhibit transcription of an operably linked promoter. By contrast, a LexA-C1 fusion protein activates transcription when bound to an operably linked Lex operator.

The expression vector further may comprise a sequence encoding a signal peptide, or signal sequence. In one embodiment, the signal peptide directs the transport of the biotin-binding protein to chloroplasts, in which some enzymes that require biotin are localized, such as acetyl-coenzyme A carboxylase. Gornicki et al., Proc. Natl. Acad. Sci. USA 91: 6860-6864 (1994). In another embodiment, the signal peptide directs export of the biotin-binding protein, where it may cause sterility by its effect on cells in the vicinity of those producing the biotin-binding protein. Plant signal sequences are reviewed in Jones et al., Tansley Review 17: 567-597 (1989). The expression vector is introduced into plant tissue by standard methods and transgenic plants are selected and propagated.

Male fertility can be restored by co-expression in the transgenic plants of a second gene that inhibits transcription of the gene encoding a biotin-binding polypeptide, or inhibits translation of its mRNA. The second gene may be provided through a cross between the male-sterile line and a second parent line that contains an expression vector comprising the restorer gene. Alternately, the expression of the gene encoding a biotin-binding polypeptide may be temporarily suppressed by inducing the expression of a suppressor gene that is linked operably to an inducible promoter. This suppressor gene may be located on the same expression vector as the gene encoding a biotin-binding polypeptide, such that the expression products of both genes are co-localized within the transformed plant cell. Alternatively, male fertility can be restored by spraying developing plants with solutions of biotin. For the purposes of the invention, male fertility is restored when the plant produces sufficient pollen to enable it to sexually reproduce.

I. Biotin-Binding Proteins and Encoding Polynucleotides

Biotin-binding proteins of the invention sequester intracellular biotin required as a co-factor by various plant enzymes. In the case of avidin and streptavidin, biotin is depleted from biotin-binding enzymes as well as from intracellular stores because of the enormously strong binding of biotin to these proteins. The K_{d} at neutral pH is about 10⁻¹⁵ M and 10⁻¹⁴ M for avidin and streptavidin, respectively. Green, Methods in Enzymology 184: 51-67 (1990). Avidin inhibits biotin-binding enzymes in general, and the inhibition of an enzyme by avidin is diagnostic that it requires biotin. Metzler, BIOCHEMISTRY, THE CHEMICAL REACTIONS OF LIVING CELLS (Academic Press, NY, 1977, page 438). Enzyme inhibition may occur through the sequestration of biotin in a complex with avidin, or through steric hindrance caused by the binding of avidin to biotin bound in the active site of the enzyme. In this regard, the ability of immobilized avidin to bind biotin-containing proteins indicates that avidin is capable of binding biotin even when biotin occupies an enzyme active site. Green, Methods in Enzymology 184: 51-67 (1990).

A biotin-binding protein of the invention thus is believed to inhibit key metabolic processes requiring enzymes with biotin co-factors, which may interfere with the function of the plant cell. Conversely, removal of the biotin-binding activity by repression of expression of the biotin-binding protein or by the exogenous addition of more biotin to the cell may result in normalized cellular function. If the biotin-binding protein is expressed in a cell that is directly or indirectly involved in gamete formation or function, disruption of cellular function may result in male- or female-sterility.

“Biotin-binding” means that the polypeptide sequence is capable of specifically interacting with biotin. The biotin-binding proteins of the invention may have a lower or higher affinity for biotin than avidin or streptavidin. Polypeptides with a higher affinity for biotin than avidin include those polypeptides that interact covalently with biotin to form a polypeptide-biotin adduct. Non-covalent binding between biotin and a polypeptide is characterized by an equilibrium between bound and unbound biotin. Polypeptides having a lower affinity for biotin than avidin will have to be present in proportionally higher concentrations to bind the same amount of biotin as avidin.

This property can be advantageous. Promoters of the invention need not be expressed exclusively in one tissue type. While promoter activity is highest in tissues that are critical for gamete formation or function, the promoter may operate at low levels in other tissue types. Because only high concentrations of the present biotin-binding proteins will be detrimental to cell viability, low-level production of biotin-binding polypeptides in tissues nonessential to gamete formation or function will be more easily tolerated. The level of expression of a biotin-binding polypeptide that is required to impact cell viability will be much higher than the level of naturally occurring biotin binding proteins in the cell.

Modified versions of naturally occurring biotin-binding polypeptides are useful for the invention, provided they bind sufficient biotin to induce sterility. One class of modified proteins useful for the invention are “muteins,” in which one or more amino acids have been inserted, deleted, or substituted from the wild-type sequence. These muteins may be more stable or more resistant to proteases than native avidin or streptavidin, for example. Furthermore, fragments of all biotin-binding proteins are envisioned, provided the fragment possesses the ability to bind biotin with an affinity sufficiently high to induce sterility.

Accordingly, the invention encompasses variants of biotin-binding proteins, where variants represent polypeptides that are modified from the naturally occurring amino acid sequences. All variants of the invention possess the ability to specifically bind biotin. The effect of modifying the naturally occurring form of a biotin-binding polypeptide can be determined by any of the routine biotin binding assays known in the art. For an example of a routine biotin-binding assay, see Green, Methods in Enzymology 184: 51-67 (1990). Typically, these assays can be conducted in vitro using polypeptides that have been produced through recombinant expression in a suitable host, such as a bacteria, using methods well know in the art. See Sambrook, et al., for example.

Biotin-binding sites are known to be structurally conserved throughout evolution. Samols et al., J. Biol. Chem. 263: 6461-6464 (1988); Gornicki et al., Proc. Natl. Acad. Sci. 91: 6860-6864 (1994). Thus, biotin-binding activity may be expected to be conferred by the structural arrangement of those amino acid residues that have been conserved in evolution. The structure of the biotin-binding regions of avidin and streptavidin is known at the atomic level through X-ray crystallographic analysis. Livnah et al., Proc. Natl. Acad. Sci. USA 90: 5076-5080 (1993); Hendrickson et al., Proc. Natl. Acad. Sci. USA 86: 2190-2194 (1989). This detailed structural knowledge provides guidance for the artisan to modify a gene encoding conserved biotin-binding sites prior to insertion and expression in a plant. Furthermore, the biotin-binding sites of numerous enzymes that use biotin as a cofactor have been identified. In the family of acetyl-coenzyme A carboxylases, for example, biotin is covalently attached to a lysine residue in a peptide consensus sequence E(V/A)MK(M/L). Gornicki et al., Proc. Natl. Acad. Sci. 91: 6860-6864 (1994).

“Substitutional variants” typically contain the exchange of one amino acid for another at one or more sites within the protein, and are designed to modulate one or more properties of the protein, such as stability against proteolytic cleavage. Substitutions preferably are conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine, glutamine, or glutamate; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine.

“Insertional variants” include fusion proteins containing amino acids not found in the native protein. Such variants include proteins comprising an N-terminal signal sequence, for instance. In many cases, signal sequences are removed co-translationally. Other variants may include proteins with covalently attached moieties that facilitate detection or purification by methods well known in the art. These protein derivatives may be particularly useful in vitro characterization of a variant to ascertain its affinity for biotin.

Yet other variants include “deletional variants,” which are fragments of biotin-binding polypeptides. By making the encoded polypeptide shorter, the level of expression of biotin-binding activity from the same promoter may be increased. Further, many plant proteins that bind biotin also catalyze enzymatic reactions, which may produce unwanted metabolic products. Fragments or subunits of biotin-binding enzymes may be produced that do not possess unwanted enzymatic activity of the holoprotein. Typically, amino acid residues may be removed from either the N-terminal or C-terminal region of a protein without causing a loss of activity. The amount of residues that may be removed from a protein depends on the particular location and structure of its biotin-binding region, although a fragment will constitute typically no less than 70% of the native sequence of the polypeptide. For example, one fragment useful for the invention comprises the sequence E(V/A)MK(M/L), which can act as a substrate for the covalent attachment of biotin. Id.

The number and location of amino acid substitutions depends on many factors. The skilled artisan appreciates that proteins in general are marginally stable, and that a large number of amino acid substitutions will result in improper folding of the polypeptide with the loss of activity. Generally, between 1 to about 20 amino acid substitutions may be made before problems are encountered with proper folding of a globular protein. Wells, Biochemistry 29:8509-8517 (1990). Moreover, the artisan appreciates that amino acids within the protein interior are particularly sensitive to variation given the compact packing of residues within the interior of proteins. Amino acid residues may be modified by methods known in the art, such as site-directed mutagenesis or alanine-scanning mutagenesis. Cunningham et al., Science 244:10811085 (1989). Routine methods of detecting biotin binding can be used to ascertain which variants retain the ability to bind biotin.

In preferred embodiments, the biotin-binding protein differs from the native sequence by no more than 50 amino acid substitutions, insertions, or deletions. In a more preferred embodiment, the biotin-binding protein differs from the native sequence by no more than 20 amino acid substitutions, insertions, or deletions. In an even more preferred embodiment, the biotin-binding protein differs from the native sequence by no more than 10 amino acid substitutions, insertions, or deletions. In an especially preferred embodiment, the biotin-binding protein differs from the native sequence by no more than 5 amino acid substitutions, insertions, or deletions. Preferred biotin-binding polypeptide fragments are at least 5, 10 or 30 contiguous amino acids in length. More preferred biotin-binding polypeptide fragments are at least 50 contiguous amino acids in length. Even more preferred biotin-binding polypeptide fragments are at least 100 contiguous amino acids in length.

Nucleotide sequences encoding biotin-binding polypeptides can be isolated by known methods. Genes encoding biotin-binding proteins may be isolated, modified and expressed at high levels in plants to confer sterility. Preferably, the biotin-binding proteins are plant proteins, although useful biotin-binding proteins may also be derived from animals or bacteria. Known plant biotin-binding proteins include: a seed-specific biotinylated protein, SBP65 (Duval et al. Biochem J. 299: 141-150 [1994]); a multisubunit acetyl coenzyme A carboxylase (Reverdatto et al. Plant Physiol. 119: 961-978 [1999]; Alban et al., Biochem. J. 300: 557-565 [1994]; Gornicki et al., Proc. Natl. Acad. Sci. USA 91: 6860-6864 [1994]); 3-methylcronotyl-coenzyme A carboxylase (Song et al., Proc. Natl. Acad. Sci. 91: 5779-5783 [1994]); carbon-dioxide ligase (Wurtele et al., Arch. Biochem. Biophys. 278: 179-186 [1990]); and pyruvate decarboxylase (Id.). These enzymes have animal and bacterial counterparts with similar, conserved biotin-binding regions.

These and other similar genes can be isolated using the polymerase chain reaction (PCR) using standard methods. See Erlich, PCR TECHNOLOGY: PRINCIPLES AND APPLICATIONS FOR DNA AMPLIFICATION (Stockton Press, NY, 1989) and Innis et al., PCR PROTOCOLS: A GUIDE TO METHODS AND APPLICATIONS (Academic Press, San Diego, 1990). Methods for PCR amplification of long nucleotide sequences, such as the ELONGASE™ system (Life Technologies, Inc., Gaithersburg, Md.) can also be used for obtaining longer nucleotide sequences, such as genomic clones. PCR primers complementary to the 5′ and 3′ termini of known gene sequences can be synthesized using commercial oligonucleotide synthesizers, such as those supplied by Applied Biosystems (Foster City, Calif.). In a preferred embodiment, the primers include additional nucleotide sequences containing restriction endonuclease cleavage sites. The presence of such sites allows for the directional cloning of PCR products into suitable cloning vectors after treatment with an appropriate restriction enzyme. See Finney, “Molecular Cloning of PCR Products” in CURRENT PROTOCOLS IN MOLECULAR BIOLOGY, Ausubel et al. Eds. (John Wiley & Sons, New York, 1987) p. 15.7.1.

These clones can be analyzed using a variety of standard techniques such as restriction analysis, Southern analysis, primer extension analysis, and DNA sequence analysis. Primer extension analysis or S1 nuclease protection analysis, for example, can be used to localize the putative start site of transcription of the cloned gene. Ausubel at pages 4.8.1-4.8.5; Walmsley et al., “Quantitative and Qualitative Analysis of Exogenous Gene Expression by the S1 Nuclease Protection Assay,” in METHODS IN MOLECULAR BIOLOGY, VOL. 7: GENE TRANSFER AND EXPRESSION PROTOCOLS, Murray (ed.), pages 271-281 (Humana Press Inc. 1991). These and related techniques well known to one skilled in the art are employed for isolation of other genes of interest, and their cloning and expression.

Once the gene of interest has been isolated, standard molecular biological techniques may be used to modify the gene to produce biotin-binding variants. These techniques are well established and include oligonucleotide-directed mutagenesis. See generally CURRENT PROTOCOLS IN MOLECULAR BIOLOGY, Ausubel et al. Eds. (John Wiley & Sons, New York, 1987), and Sambrook et al., MOLECULAR CLONING: A LABORATORY MANUAL, Second Edition, (Cold Spring Harbor Laboratory Press, Cold Spring Harbor, N.Y. 1989). Individual amino acids of biotin-binding proteins may inserted, deleted or substituted, and the modified polypeptide may be expressed recombinantly and assayed for biotin-binding activity, as described above.

The gene also may be modified without changing the sequence of encoded amino acids in the polypeptide. Many amino acids are encoded by more than one codon, and different organisms exhibit distinct preferences for particular codons. Gene expression may be enhanced by tailoring the choice of codons for a particular amino acid to reflect the host organisms codon preferences. It is expected that those genes derived from plants will not require codon optimization to boost expression levels.

II. Expression Vectors and Transgenic Plants

Once a gene encoding a biotin-binding polypeptide has been isolated, it is placed into an expression vector by standard methods. See Sambrook et al., supra. The selection of an appropriate expression vector will depend upon whether the polypeptide will be expressed in a bacterial host for subsequent expression and in vitro analysis, or whether the polypeptide is to be expressed in a plant cell. Typically, an expression vector useful for generating a male-sterile plant contains: (1) prokaryotic DNA elements coding for a bacterial replication origin and an antibiotic resistance gene to provide for the growth and selection of the expression vector in the bacterial host; (2) a cloning site for insertion of an exogenous DNA sequence, for example a gene encoding the biotin-binding protein; (3) eukaryotic DNA elements that control initiation of transcription of the exogenous gene; (4) DNA elements that control the processing of transcripts, such as a transcription termination/polyadenylation sequence; and (5) a gene encoding a marker protein (e.g., a reporter gene), which is operably linked to the DNA elements that control eukaryotic transcription initiation. Additionally, the expression vector may comprise a DNA sequence encoding a signal sequence operably linked to the exogenous DNA sequence, to cause intracellular transport of the biotin-binding protein to an organelle or to cause extracellular transport. General descriptions of plant expression vectors and reporter genes can be found in Gruber et al., “Vectors for Plant Transformation,” in METHODS IN PLANT MOLECULAR BIOLOGY AND BIOTECHNOLOGY, Glick et al. (eds.), pages 89-119 (CRC Press, 1993).

(a) Tissue-Selective Promoters

In a preferred embodiment, the promoter of the expression vector has selectively higher activity in tissues that are critical for gamete formation or function. Promoters that are selectively expressed in cells critical to pollen formation or function are known in the art and include the anther-selective promoter from the Ms*5126 gene. See U.S. Pat. No. 5,689,051. Other anther-selective promoters include those from three nuclear encoded mitochondrial complex I (nCI) genes from Arabidopsis thaliana. Zabalete et al., Plant Journal 15:49-59 (1998). These promoters contain common conserved regions that confer anther-selective expression. The artisan would be guided in finding additional anther-selective promoters by searching for promoter elements that were structurally conserved with these elements. Another useful promoter is an anther-selective promoter of an Arabidopsis gene, which drives tissue-selective expression in Nicotiana tabacum. Twell et al., Sexual Plant Reprod. 6:217-224 (1993). Yet another useful promoter is an anther-selective promoter from the Brassica campestris Bgp1 gene, which may drive tissue-selective expression in transgenic A. thalania or N. tabacum. Xu et al., Molec. Gen. Genet. 239:58-65 (1993).

A tissue-selective promoter of the invention may include the entire upstream promoter region of a gene that is selectively expressed, or it may be a composite promoter. Composite promoters combine a core promoter from a gene operably linked to a regulatory region from a tissue-selective promoter that can confer tissue-selective expression. Such composite promoters are described in U.S. Pat. No. 5,962,769.

A core promoter contains essential nucleotide sequences for promoter function, including the TATA box and start of transcription. A core promoter may or may not have detectable activity in the absence of specific sequences that may enhance the activity or confer tissue-selective activity. For example, the SGB6 core promoter consists of about 38 nucleotides 5′ of the transcriptional start site of the SGB6 gene, while the Cauliflower Mosaic Virus (CaMV) 35S core promoter consists of about 33 nucleotides 5′ of the transcriptional start site of the 35S genome.

A tissue-selective regulatory region is a DNA sequence that, when operably linked to a gene, directs a higher level of transcription in a particular tissue. The SGB6 anther-selective promoter, for example, can direct preferential expression of a foreign gene in anther tissue, but not in root tissue or coleoptile tissue. A tissue-selective regulatory region that causes anther-selective expression is referred to as an “anther box.” More generally, such regions are termed “enhancers,” because they possesses the functional characteristics of an enhancer element. The combination of a tissue-selective enhancer and a core promoter can stimulate gene expression to a greater extent than a core promoter alone. This is true even in the case of a composite anther-selective promoter containing an anther box and a core promoter derived from different genes. Core promoters that are particularly useful for the invention are described in U.S. Pat. No. 5,962,769.

A particularly preferred anther-selective promoter is the promoter from the Ms*5126 gene, which was isolated from the maize inbred B73 line. The Ms*5126 promoter initiates the expression of a foreign gene from quartet to early uninucleate microspore-stage anthers. Another suitable anther-selective promoter is the SGB6 promoter, which was isolated from the maize inbred W22 line. The SGB6 promoter can induce expression of a foreign gene in anther tapetal cells from the quartet stage to the mid-uninucleate stage of microspore development. Yet another suitable anther box is obtained from the maize G9 promoter, which initiates gene expression during the meiotic to quartet stages of development. These anther boxes are described in detail in U.S. Pat. No. 5,962,769.

Promoters that are selectively expressed in cells critical to female gamete formation or function also are known in the art. It is known, for example, that papillar cells or the secretory zone of stigma can be functionally impaired by the tissue-selective expression of a dominant negative gene. For example, impairment of the secretory zone may allow pollen germination on the stigma that does not lead to the formation of seeds. Goldman et al., EMBO J. 13: 2976-2984 (1994). Thus, in one embodiment, promoters that are useful for making a female-sterile plant drive the expression of the gene encoding the biotin-binding protein selectively in stigma tissues. Useful promoters for affecting female-sterility are described in Goldman et al. and Kandasamy et al., Plant Cell 5: 263-275 (1993), among others. See also Day et al., Trends Plant Sci. 2: 106-111 (1997). The nuc1 gene is specifically expressed in the nucellus; therefore, the promoter from this gene also could be used for a female-sterile plant. See Dean et al., Plant Mol. Biol. 31: 877-886 (1996).

It is within the skill of the art to discover new promoters having the tissue-selective expression patterns that are useful for the invention. One routine method used to determine the tissue expression pattern of a promoter is a so-called “enhancer trap” experiment. Briefly, a construct, such as a transposon, that contains a reporter gene is transformed into a plant. Transcription of the reporter gene may be activated by cis-acting enhancer regions near the point of insertion of the construct, and the tissue specificity of the enhancer may be determined indirectly by assaying the expression of the reporter gene. Interesting enhancers then may be cloned by using the reporter gene as a tag. See Sundaresan et al., Genes Dev. 9: 1797-1810 (1995).

Not all promoters that function selectively in reproductive tissue will be useful for the invention. For instance, a dominant negative gene linked to a promoter from the S Locus glycoprotein of the Brassica self-incompatibility locus causes female infertility following self-pollination, but not following pollination from wild-type plants. Thorsness et al., Plant Cell 5: 253-261 (1993). It is within the skill of the artisan to choose those promoters that are suited for creation of the male- and female-sterile plants of the invention.

(b) Inducible Promoters

In some embodiments of the invention, an inducible promoter is used to allow inducible regulation of expression. For practice of this embodiment, a promoter is used that only responds to a specific external stimulus. An example of such an inducible promoter is the glutathione S-transferase system in maize. See Wiegand et al., Plant Mol. Biol. 7: 235 (1986). Yet another example is another chemical-inducible gene expression system, using the In2-2 promoter, which is activated by benzenesulfonamide herbicide safeners. De Veylder et al., Plant Cell Physiol. 38: 568-577 (1977). Yet other examples include the copper-inducible promoter described by Mett et al. (1993) Proc. Natl. Acad. Sci. USA 90: 4567-4571 and the steroid inducible promoter described by Lloyd et al. (1994) Science 266: 436-439.

(c) Selectable Markers

The expression may comprise a selectable or screenable marker. Many of the commonly used positive selectable marker genes for plant transformation were isolated from bacteria and code for enzymes that metabolically detoxify a selective chemical agent which may be an antibiotic or a herbicide. Other positive selective marker genes encode an altered target which is insensitive to the inhibitor.

Selectable marker genes include herbicide resistance genes. For example, such genes may confer resistance to phosphinothricine, glyphosate, sulfonylureas, atrazine, or imidazolinone. Preferably, the selectable marker gene is the bar gene or pat gene which encodes phosphinothricin acetyltransferase. The nucleotide sequences of bar genes can be found in EP 242246 (1987), and in White et al., Nucleic Acids Res. 18: 1062 (1990). Wohlleben et al., Gene 70: 25 (1988), disclose the nucleotide sequence of the pat gene. Bar or pat gene expression confers resistance to herbicides such as glufosinate (sold as Basta® and Ignite®, among others) and bialaphos (sold as Herbiace® and Liberty®).

Another commonly used selectable marker gene for plant transformation is the neomycin phosphotransferase II (nptII) gene, isolated from Tn5, which when placed under the control of plant regulatory signals confers resistance to kanamycin. Fraley et al., Proc. Natl Acad. Sci. U.S.A. 80: 4803 (1983). Another commonly used selectable marker is the hygromycin phosphotransferase gene which confers resistance to the antibiotic hygromycin. Vanden Elzen et al., Plant Mol. Biol. 5: 299 (1985). Additional positive selectable marker genes of bacterial origin that confer resistance to antibiotics include gentamycin acetyl transferase, streptomycin phosphotransferase, aminoglycoside-3′-adenyl transferase and the bleomycin resistance determinant. Hayford et al., Plant Physiol. 86: 1216 (1988); Jones et al., Mol. Gen. Genet. 210: 86 (1987); Svab et al., Plant Mol. Biol. 14: 197 (1990), Hille et al., Plant Mol. Biol. 7: 171 (1986).

Other positive selectable marker genes for plant transformation are not of bacterial origin. These genes include mouse dihydrofolate reductase, plant 5-enolpyruvylshikimate-3-phosphate synthase and plant acetolactate synthase. Eichholz et al., Somatic Cell Mol. Genet. 13: 67 (1987); Shah et al., Science 233: 478 (1986); Charest et al., Plant Cell Rep. 8: 643 (1990).

Another class of marker genes for plant transformation requires screening of presumptively transformed plant cells, instead of direct genetic selection of transformed cells for resistance to a substance like an antibiotic. These genes are particularly useful to quantify or to visualize the spatial pattern of expression of a gene in specific tissues and are frequently referred to as reporter genes because they can be fused to a gene or gene regulatory sequence for the investigation of gene expression.

Commonly used genes for screening presumptively transformed cells include β-glucuronidase (GUS), β-galactosidase, luciferase, green fluorescence protein, and chloramphenicol acetyltransferase. Jefferson, Plant Mol. Biol. Rep. 5: 387 (1987); Teeri et al., EMBO J. 8: 343 (1989); Koncz et al., Proc. Natl Acad. Sci. U.S.A. 84: 131 (1987); De Block et al., EMBO J. 3: 1681 (1984). Another approach to the identification of relatively rare transformation events has been use of a gene that encodes a dominant constitutive regulator of the Zea mays anthocyanin pigmentation pathway. Ludwig et al., Science 247: 449 (1990).

(d) Signal Sequences

The expression vectors may comprise the gene encoding a biotin-binding protein operably linked to a DNA sequence that codes for a peptide signal sequence. See Hones and Robinson, supra. The vector is made such that a signal sequence is fused to the N-terminal of the mature protein sequence, allowing for normal cellular processing to cleave accurately the protein molecule and yield mature, active biotin-binding protein. In one embodiment, the signal sequence is the barley alpha amylase export signal sequence. Rogers, J. Biol. Chem. 260: 3731-3738 (1985). Other preferred signal sequences are those that transport the biotin-binding protein to organelles containing essential enzymes that require biotin, such as mitochondria or chloroplasts. Transport to intracellular organelles advantageously will concentrate the biotin-binding protein locally, causing cellular disruption at lower levels of expression.

(e) Transformation

Expression vectors containing a gene encoding a biotin-binding protein can be introduced into protoplasts, or into intact tissues, such as immature embryos and meristems, or into callus cultures, or into isolated cells. Preferably, expression vectors are introduced into intact tissues. General methods of culturing plant tissues are provided, for example, by Miki et al., “Procedures for Introducing Foreign DNA into Plants,” in METHODS IN PLANT MOLECULAR BIOLOGY AND BIOTECHNOLOGY, Glick et al. (eds.), pages 67-88 (CRC Press, 1993), and by Phillips et al., “Cell/Tissue Culture and In Vitro Manipulation,” in CORN AND CORN IMPROVEMENT, 3rd Edition, Sprague et al. (eds.), pages 345-387 (American Society of Agronomy, Inc. et al. 1988).

Techniques for transforming a wide variety of higher plant species are well known and described in the technical, scientific, and patent literature. See, for example, Weising et al., Ann. Rev. Genet. 22: 421-477 (1988). For example, the DNA construct may be introduced directly into the genomic DNA of the plant cell using techniques such as electroporation, polyethylene glycol (PEG), particle bombardment, silicon fiber delivery, or microinjection of plant cell protoplasts or embryogenic callus. See, e.g., Tomes, et al., “Direct DNA Transfer into Intact Plant Cells Via Microprojectile Bombardment,” in PLANT CELL, TISSUE AND ORGAN CULTURE, FUNDAMENTAL METHODS, Gamborg, et al. (eds.), pages 197-213 (Springer-Verlag, 1995); and U.S. Pat. No. 5,990,387. The introduction of DNA constructs using PEG precipitation is described in Paszkowski et al., Embo J. 3: 2717-2722 (1984). Electroporation techniques are described in Fromm et al., Proc. Natl. Acad. Sci. (USA) 82: 5824 (1985). Ballistic transformation techniques are described in Klein et al., Nature 327: 70-73 (1987).

Agrobacterium tumefaciens-mediated transformation techniques are well described in the scientific literature. See, for example Horsch et al., Science 233: 496-498 (1984); Fraley et al., Proc. Natl. Acad. Sci. (USA) 80: 4803 (1983); and, PLANT MOLECULAR BIOLOGY: A LABORATORY MANUAL, Clark (ed.), Chapter 8, (Springer-Verlag, 1997). The DNA constructs may be combined with suitable T-DNA flanking regions and introduced into a conventional Agrobacterium tumefaciens host vector. The virulence functions of the Agrobacterium tumefaciens host will direct the insertion of the construct and adjacent marker into the plant cell DNA when the cell is infected by the bacteria. See U.S. Pat. No. 5,591,616. Although Agrobacterium is useful primarily in dicots, certain monocots can be transformed by Agrobacterium. For instance, Agrobacterium transformation of maize is described in U.S. Pat. No. 5,550,318.

Other methods of transfection or transformation include Agrobacterium rhizogenes-mediated transformation (see, e.g., Lichtenstein et al. in GENETIC ENGINEERING, Rigby (ed.) vol. 6 (Academic Press, 1987); and Lichtenstein et al. in DNA CLONING, Glover (ed.), Vol. II, (IRI Press, 1985). WO 88/02405 describes the use of A. rhizogenes strain A4 and its Ri plasmid along with A. tumefaciens vectors pARC8 or pARC16. Liposome-mediated DNA uptake is described in Freeman et al., Plant Cell Physiol. 25: 1353 (1984)), and the vortexing method is described in Kindle, Proc. Natl. Acad. Sci. (USA) 87: 1228 (1990).

DNA can also be introduced into plants by direct DNA transfer into pollen as described by Zhou et al., Methods in Enzymology, 101:433 (1983); D. Hess, Intern Rev. Cytol. 107:367 (1987); and Luo et al., Plant Mol. Biol. Reporter 6:165 (1988). Expression of polypeptide coding genes can be obtained by injection of the DNA into reproductive organs of a plant as described by Pena et al., Nature 325:274 (1987). DNA can also be injected directly into the cells of immature embryos and the rehydration of desiccated embryos as described by Neuhaus et al., Theor. Appl. Genet. 75:30 (1987); and Benbrook et al. in Proceedings Bio Expo 1986, Butterworth, Stoneham, Mass., pp. 27-54 (1986). A variety of plant viruses that can be employed as vectors are known in the art and include cauliflower mosaic virus (CaMV), geminivirus, brome mosaic virus, and tobacco mosaic virus.

Suitable methods for corn transformation are provided by U.S. Pat. No. 5,886,244, and Fromm et al., Bio/Technology 8: 833 (1990). Standard methods for the transformation of rice are described by Christou et al., Trends in Biotechnology 10: 239 (1992), and by Lee et al., Proc. Nat'l Acad. Sci. USA 88: 6389 (1991). Wheat can be transformed using methods that are similar to the techniques for transforming corn or rice. Furthermore, Casas et al., Proc. Nat'l Acad. Sci. USA 90: 11212 (1993), describe a method for transforming sorghum, while Wan et al., Plant Physiol. 104: 37 (1994), describe a method for transforming barley.

In general, direct transfer methods are preferred for the transformation of a monocotyledonous plant, particularly a cereal such as rice, corn, sorghum, barley or wheat. Suitable direct transfer methods include microprojectile-mediated delivery, DNA injection, electroporation, and the like. See, for example, Gruber et al., supra, Miki et al., supra, and Klein et al., Bio/Technology 10: 268 (1992). More preferably, expression vectors are introduced into tissues of a monocotyledonous plant using microprojectile-mediated delivery with a biolistic device. It is understood in the art that expression vectors are linearized upon stable integration in the host genome.

III. Production of Male-Sterile Plants

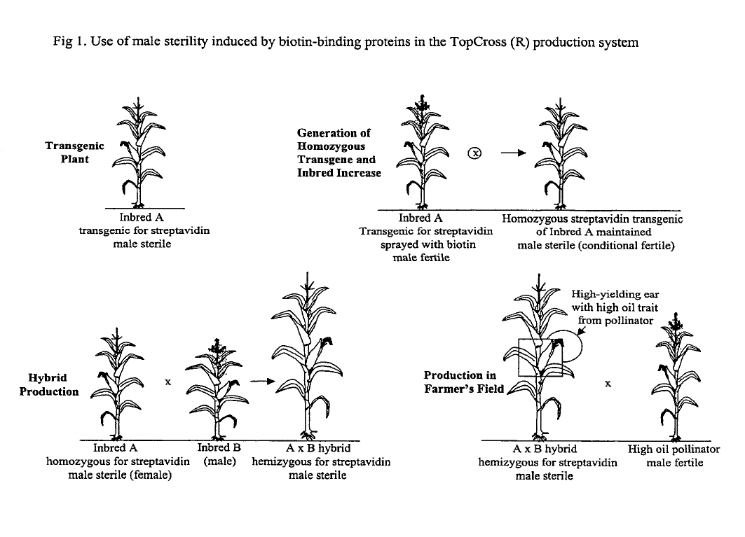

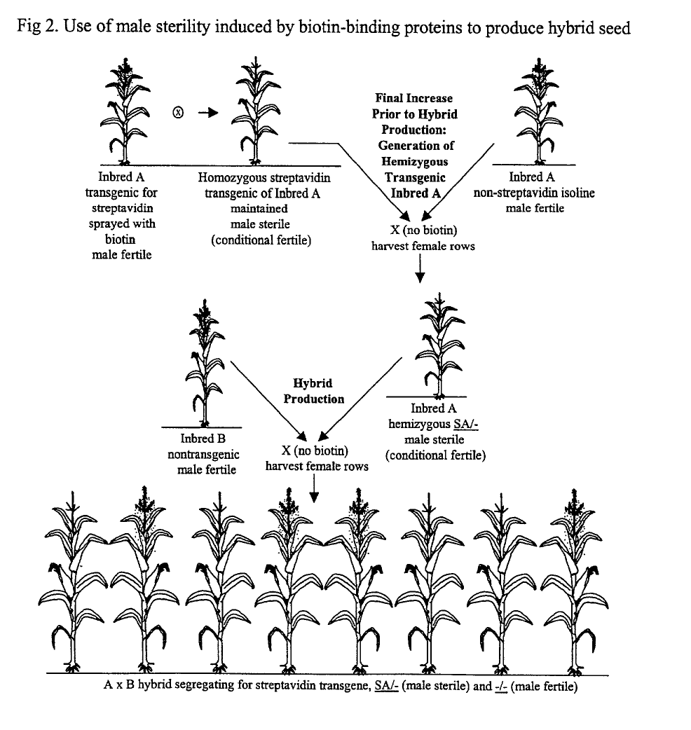

Male-sterile plants may be made as a step in a method for F1 hybrid production. If the male-sterile inbred is homozygous for the male-sterile trait, it may be used in the TopCross® method. See FIG. 1 and U.S. Pat. No. 5,706,603. If the male-sterile plant is hemizygous for the male-sterile trait, it may be used for conventional F1 hybrid seed production. See FIG. 2.

To induce sterility pursuant to the present invention, an expression vector is constructed in which a DNA sequence encoding a biotin-binding polypeptide is operably linked to DNA sequences that regulate gene transcription in plant tissues. The general requirements of an expression vector are described above. To achieve sterility, it is preferred that the biotin-binding polypeptide is expressed during the reproductive stage in the adult plant.

Mitotic stability can be achieved using plant viral vectors that provide epichromosomal replication. A preferred method of obtaining mitotic stability is provided by the integration of expression vector sequences into the host chromosome. Such mitotic stability can be provided by the microprojectile delivery of an expression vector to plant tissue, or by using other standard methods as described above. See, for example, U.S. Pat. No. 5,886,244.

IV. Restoration of Male Fertility in the F1 Hybrid

The above-described methods can be used to produce transgenic male-sterile plants for the production of F1 hybrids in large-scale directed crosses between inbred lines. If all egg cells of the transgenic male-sterile plants do not contain the recombinant gene encoding a biotin-binding protein, then a proportion of F1 hybrids will have a male-fertile phenotype. On the other hand, F1 hybrids will have a male-sterile phenotype if the recombinant gene encoding a biotin-binding protein is present in all egg cells of the transgenic male-sterile plants. Thus, it might be desirable to use a male fertility restoration system to provide for the production of male-fertile F1 hybrids. Such a fertility restoration system has particular value in autogamous species when the harvested product is seed.

The commonly proposed approach to restoring fertility in a line of transgenic male-sterile plants requires the production of a second “restorer” line of transgenic plants. For example, see Mariani et al., Nature 357: 384 (1992). In the present case, an analogous approach would require the production of a restorer line of transgenic plants that express gene products that specifically target and repress expression of the gene encoding a biotin-binding protein. Preferred gene products with this desired function are described below.

As an alternative to the use of a restorer line, a transgenic male-sterile line can be self-pollinated following repression of the gene encoding a biotin-binding protein. For example, an inducible promoter may control the expression of the gene product that specifically targets and represses expression of the gene encoding a biotin-binding protein. The male-sterile plant is treated with the inducer, the gene product is expressed, and the mRNA of the gene encoding a biotin-binding protein is inactivated, leading to restored male fertility.

As yet another alternative approach to restoring male fertility, the action of biotin-binding proteins may be overcome by increasing the expression of biotin. Such a method is described by Patton in International publication WO 96/17944. This method provides a transgenic plant expressing a biotin biosynthetic enzyme that produces high levels of biotin. This transgenic plant line may be crossed with a presently described biotin-binding protein producing male-sterile plant to reverse sterility.

Alternately, ribozymes can be designed to express endonuclease activity that is directed to a certain target sequence in a mRNA molecule. For instance, inheritably integrated ribozymes targeted against potato spindle tuber viroid RNA have been shown to confer resistance against viroid replication. Yang et al., Proc. Natl. Acad. Sci. USA 94: 4861-4865 (1997). For repressing the expression of Δ9 desaturase, a multimeric hammerhead ribozyme recently has been shown to be more efficient than non-enzymatic antisense molecules. Merlo et al., Plant Cell 10: 1603-1621 (1998); cf. Knutzon et al., Proc. Natl. Acad. Sci. USA 89: 2624-2628 (1992).

RNA synthesis, processing, and transport from the nucleus have been found to be highly structured and locally organized in vivo, and the in vivo efficacy and predictability of ribozyme and antisense technology is enhanced by co-localized expression of the ribozyme or other antisense molecule and the target of interest. Reviewed by Arndt et al., Genome 40: 785-797 (1997). Co-localized expression can be accomplished in a number of ways, including the use of RNA localization signals on the target and ribozyme molecules. Alternately, the expression vector may comprise a ribozyme gene in a cis configuration with a gene encoding a biotin-binding protein. The expression of the gene encoding a biotin-binding protein and the ribozyme could be regulated separately, such that the gene encoding a biotin-binding protein is expressed to cause sterility, and the ribozyme is expressed in response to an inducer to restore male fertility. This inducer may be provided by crossing the plant with a restorer line or by spraying the plant with the appropriate inducer.

In a similar approach, fertility can be restored by the use of an expression vector containing a nucleotide sequence that encodes an antisense RNA. The binding of antisense RNA molecules to target mRNA molecules results in hybridization arrest of translation. Paterson et al., Proc. Natl. Acad. Sci. USA 74: 4370 (1987). In the context of the present invention, a suitable antisense RNA molecule would have a sequence that is complementary to the mRNA encoding a biotin-binding protein. In a preferred embodiment of the invention, the antisense RNA is under the control of an inducible promoter. Activation of this promoter then allows restoration of male fertility. Schmülling et al., Mol. Gen. Genet. 237: 385-394 (1993).

In a further alternative approach, expression vectors can be constructed in which an expression vector encodes RNA transcripts capable of promoting RNase P-mediated cleavage of mRNA molecules. According to this approach, an external guide sequence is constructed for directing the endogenous ribozyme, RNase P, to mRNA encoding a biotin-binding protein, which is subsequently cleaved by the cellular ribozyme. Altman et al., U.S. Pat. No. 5,168,053; Yuan et al., Science 263: 1269 (1994). Preferably, the external guide sequence comprises a ten to fifteen nucleotide sequence complementary to the target mRNA, and a 3′-NCCA nucleotide sequence, wherein N is preferably a purine. Id. The external guide sequence transcripts bind to the targeted mRNA species by the formation of base pairs between the mRNA and the complementary external guide sequences, thus promoting cleavage of mRNA by RNase P at the nucleotide located at the 5′-side of the base-paired region. Id.

In an alternative method for restoring male fertility, transgenic male-sterile plants contain an expression vector that, in addition to a promoter sequence operably linked to a gene encoding a biotin-binding protein, also contains a prokaryotic regulatory element. Transgenic male-fertile plants are produced that express a prokaryotic polypeptide under the control of the promoter. In the F1 hybrids, the prokaryotic polypeptide binds to the prokaryotic regulatory sequence and represses the expression of the gene encoding the biotin-binding protein.

For example, the LexA gene/LexA operator system can be used to regulate gene expression pursuant to the present invention. See U.S. Pat. No. 4,833,080 (“the '080 patent) and Wang et al., Mol. Cell. Biol. 13: 1805 (1993). More specifically, the expression vector of the male-sterile plant would contain the LexA operator sequence, while the expression vector of the male-fertile plant would contain the coding sequences of the LexA repressor. In the F1 hybrid, the LexA repressor would bind to the LexA operator sequence and inhibit transcription of the gene encoding the biotin-binding protein.

LexA operator DNA molecules can be obtained, for example, by synthesizing DNA fragments that contain the well-known LexA operator sequence. See, for example, the '080 patent and Garriga et al., Mol. Gen. Genet. 236: 125 (1992). The LexA gene may be obtained by synthesizing a DNA molecule encoding the LexA repressor. Gene synthesis techniques are discussed above and LexA gene sequences are described, for example, by Garriga et al., supra. Alternatively, DNA molecules encoding the LexA repressor may be obtained from plasmid pRB500, American Type Culture Collection accession No. 67758.

Those of skill in the art can readily devise other male fertility restoration strategies using prokaryotic regulatory systems, such as the lac repressor/lac operon system or the trp repressor/trp operon system.

Still another method for restoring fertility is to spray developing plants with a solution of biotin. The biotin solution may comprise a minimum amount of an organic co-solvent such as DMSO to ensure complete solubility of the biotin. However, the biotin solution may contain no organic co-solvent. Spraying may commence as early as the meiotic phase of pollen development. Spraying may commence later in pollen development as well. Spraying is generally repeated at regular intervals until pollen shed is observed. The intervals between spraying will vary between 1 and 7 days. In a preferred embodiment of the invention, spraying is repeated every 3 to 5 days.

V. Controlling Female Fertility

All the hybrid crossing schemes described above require the harvest of female rows; that is, hybrid seed are harvested only from the male-sterile plants. It would be advantageous to include the male rows in the harvest as well, provided the males were female-sterile and did not produce seed through selfing. This advantageously would reduce harvesting costs and would reduce the overall cost of producing hybrid seed by more efficiently using the land required for hybrid seed production. To this end, the control of female fertility in the male plants would be desirable.

This can be accomplished by techniques similar to those used to generate tissue-selective expression in cells critical for pollen formation or function. Namely, a promoter can be operably linked to a gene encoding a biotin-binding protein, where the promoter is selectively expressed in tissues that are critical for the formation or function of female gametes or that are critical for successful pollen germination. Selective expression of the promoter may be conferred by coupling the promoter to one or more enhancer regions that are selectively expressed in such tissues. Promoter activity may be augmented or replaced by an inducible promoter. In this embodiment, the production of seeds is disrupted or arrested by spraying the plant with an inducer that activates the inducible promoter.

To restore female fertility, female-sterility can be suppressed in the same manner as male-sterility, by repressing the detrimental effects of the biotin-binding protein on cellular viability. This can be accomplished by using any of the methods for reversing male-sterility, described above. For example, female fertility can be restored by crossing the female-sterile line with a restorer line that comprises a gene whose gene product represses the expression of the gene encoding the biotin-binding protein, or the female-sterile plant can be sprayed with biotin to restore conditionally fertility.

VI. TopCross® Method of Seed Production

Hybrid seeds with one or more desired grain or seed traits can be advantageously produced using the male-sterile plant lines of the instant invention. A transgenic male-sterile line carrying a gene coding a biotin-binding polypeptide is crossed as the male-sterile female parent to a male-fertile pollinator plant which carries one or more genes for a desired grain or seed trait. Hybrid seeds having the desired grain or seed trait produced by means of this method are harvested.

The preferred ratio of male-sterile to male-fertile pollinator parent seeds planted in the seed production field depends on the genetics of each parent and is varied in order to optimize yield. The ratio of male-sterile to male-fertile pollinator parent ranges from approximately 6:1 to approximately 9:1. Preferably, the ratio of male-sterile to male-fertile pollinator parent is approximately 8:1. Most preferably, the ratio of male-sterile to male-fertile pollinator parent is approximately 9:1.

(a) Seed Production

The male-fertile pollinator plant line preferably is homozygous for the gene(s) controlling the desired grain or seed trait. The method for production of hybrid seeds in which a male-sterile parent is crossed to a male-fertile pollinator line that is homozygous for one or more genes for a desired grain or seed trait is sometimes referred to as the TopCross® method. See U.S. Pat. Nos. 5,706,603 and 5,704,160.

(b) Choice of Lines

The male-sterile and male-fertile lines crossed to make the hybrid seeds of the instant invention can be any compatible combination of hybrid, inbred or synthetic lines. A transgenic line carrying the gene encoding the biotin-binding protein operably linked to a constitutive or inducible promoter is produced using the methods described above. The male-sterile line may carry one or more copies of the gene encoding the biotin-binding protein.

The male-sterile and male-fertile pollinator lines utilized for production hybrid seeds with a desired grain or seed trait, according to the methods of the instant invention, are inbred lines, hybrid lines, synthetic open pollinated lines or genetic stock. The inbred lines, hybrid lines, synthetic open pollinated lines or genetic stock are produced by any of the methods well known to the skilled plant breeder. See, for example, J. M. Poehlman, BREEDING FIELD CROPS, 3rd ed. (Van Nostrand and Reinhold, New York, N.Y., 1987). Test crosses among selected inbred and/or hybrid lines are made to evaluate specific combining ability. Commercially acceptable combinations are identified.

A male-fertile pollinator plant line is selected which (1) carries one or more genes for a desired grain or seed trait and (2) is compatible with the male-sterile line to which it will be crossed. The gene(s) controlling the selected grain trait or seed trait may be dominant so that the trait is readily expressed in the hybrid seeds. However, the gene(s) controlling the selected grain trait or seed trait may be recessive. In this event, the male-sterile line and the male-fertile pollinator line each are made homozygous for the gene(s) so that the desired phenotype is expressed in all seed produced by the cross. Accordingly, the male-sterile and male-fertile pollinator lines may each carry genes which control a grain or seed trait in progeny seeds. The gene(s) for the desired grain or seed trait are introduced into the male-sterile line or the male-fertile pollinator line by traditional breeding methods and/or genetic engineering techniques.

The desired grain or seed trait is a nutritional or physiological characteristic of the seed which significantly affects industrial type, germination or disease resistance. The desired grain or seed trait may impact such characteristics as oil, protein and starch content, as well as protein quality and starch type, among others. The methods of the instant invention are used to produce specialized seed or grain types which are used in different markets. High oil corn, for example, is used to replace animal fats added to livestock feed. High amylose corn is used to make adhesives, degradable plastic films and packaging material. Starch from waxy corn is used in many different foods such as soups and puddings.

The male-fertile pollinator line may carry useful alleles of one or more genes that affect starch and protein characteristics. Genes which affect starch and protein characteristics include, but are not limited to, sugary (su), amylose-extender (ae), brittle (bt), dull (du), floury (fl), opaque (o), horny (h), shrunken (sh) and waxy (wx). See, for example, Hannah et al., Sci. Hortic. 55: 177-197 (1993). The properties of starch obtained from maize plants homozygous recessive for ae, du, wx, ae and aewx have been characterized. See Brockett et al., Starch/Starke 40: 175-177 (1988) and U.S. Pat. No. 5,516,939.

Alternatively, the pollinator line may be transformed with a gene that affects starch content. For example, the male-fertile pollinator may be transformed with a bacterial gene encoding ADP-glucose pyrophosphorylase which is active in the presence of metabolites which inhibit the plant enzyme. The resulting seed has a higher starch content. See Sivak et al., J. of Environ. Polymer Degradation 3(3): 145-152 (1995).

The pollinator line may carry one or more genes that affect fatty acid content. For example, the fan1 gene controls low linolenic fatty acid in soybean. Hammond et al., Crop Sci. 231:192 (1993). The fas^{a} allele confers high stearic fatty acid content in soybean. Graef et al., Crop Sci. 25: 1076 (1985). The fap1 and fap2 alleles confer low palmitic acid content and high palmitic acid content, respectively, in soybean. Erickson et al., Crop Sci. 18: 644 (1988).

The pollinator line may be transformed with a gene which affects seed oil content. For example, the pollinator line may carry a gene encoding stearoyl-acyl carrier protein (stearoyl-ACP) desaturase which catalyzes the first desaturation step in seed oil biosynthesis. The stearoyl-ACP desaturase gene may be operably linked to a seed-specific promoter. Plants such as sunflower, maize, canola or soybean transformed with the stearoyl-ACP desaturase gene produce altered stearic acid levels and can be used to produce seed oil containing modified or altered levels of saturated and unsaturated fatty acids. See, for example, U.S. Pat. No. 5,443,974.

Alternatively, the pollinator line may carry an antisense gene which inhibits the expression of a target gene in the biosynthetic pathway of the grain or seed trait. For example, the pollinator line carries an antisense gene that inhibits the expression of stearoyl-acyl desaturase. The antisense gene may be operably linked to a seed-specific promoter. Plants transformed with the stearoyl-ACP desaturase antisense gene produce increased stearate levels in seeds. See, for example, Knutzon et al., Proc. Natl. Acad. Sci. USA 89: 2624-2628 (1992).

(c) Production of Male-Sterile Hemizygous Lines

It often is advantageous to produce a plant that is homozygotic for a dominant gene that confers male sterility. Classically, this requires vegetative or clonal propagation of the hemizygotic female line. However, clonal propagation is expensive, which limits conunercial utility of dominant sterility systems. To avoid clonal propagation, male sterility may be suppressed in the hemizygote to produce a “conditional fertile” plant. Suppression of male sterility may be accomplished by spraying with biotin or an inducer of a negative regulator gene, as described above. The conditional fertile plant may then be selfed to produce inbred progeny that are homozygotic for the gene encoding the biotin-binding protein. Generation of such a homozygote (“Inbred A”) is diagramed in the first row of plants in FIG. 1.

As shown in the second row of plants in FIG. 1, crossing Inbred A with another inbred plant not containing the gene encoding the biotin-binding protein (“Inbred B”) produces a male-sterile plant that is hemizygotic for the gene encoding the biotin-binding protein (“A×B hybrid”). By combining favorable genetic characteristics from the two inbred parent lines, the hybrid offspring typically are more vigorous, as reflected in growth rate, improved resistance, etc. The relative strength of the hybrid is referred to as “hybrid vigor.”

For commercial production using the TopCross® method, the A×B hybrid may then be crossed with a third inbred line that possesses a desired trait, such as high oil yield, as shown in FIG. 1. The progeny of this cross will benefit from the improved vigor of the hybrid as well as the improved trait conferred by the third inbred line. Seed harvested from the progeny will have a high yield of oil, making them useful as feed or as a source of oil. Alternately, the seeds may be planted to produce more progeny.

The gene encoding the biotin-binding protein will segregate 1:1 in the progeny of the cross with the third line. Because the gene encoding the biotin-binding protein is dominant, 50% of the resulting progeny will be male-sterile. For species that reproduce through wind pollination, such as corn, this number of male-sterile plants is not a practical impediment to producing seed, because the 50% of the progeny that are male-fertile produce enough pollen to ensure pollination of all the plants in the field.

(d) Trait Stacking

In the TopCross® method described above, two inbred lines, one of which is homozygous for the gene encoding the biotin-binding protein, are crossed to obtain the A×B hybrid. As described above, desirable traits of Inbred A or Inbred B will be hemizygotic in the A×B hybrid and will be further segregated in its progeny. If a beneficial phenotype were realized fully only in the homozygote, the TopCross® method would result in diminishing the favorable properties in the progeny.

Alternatively, it would be desirable to start with a parent line for commercial production that was hemizygotic for the gene encoding the biotin-binding protein, but homozygotic for alleles for one or more desirable traits (“Inbred A: Hemizygous BBP” of FIG. 2). A cross between Inbred A: Hemizygous BBP and another inbred line possessing a desired trait, such as high oil yield (“Inbred B” of FIG. 2), will produce seed that are heterozygotic for the desirable alleles.

As indicated in FIG. 2, half the resulting progeny will be hemizygotic for the gene encoding the biotin-binding protein. Homozygosity can be restored in these hemizygotes by repressing male-sterility, as previously described, followed by selfing. Restoration of homozygosity can be screened not only for all the desired alleles of the parent, but also for the desired trait added by the cross with Inbred B. In this manner, desirable, complementary traits can be stacked into a single line, provided recombination brings the stacked genes into the same transgenic locus. Further, the resulting inbred line will be homozygotic for the gene encoding the biotin-binding protein, and will be available as a parent line in a reiterative cross with a male-fertile isoline, as shown in the top row of FIG. 2. In this manner, this breeding scheme may be applied recursively to stack desirable traits.

For this method, “isolines” of Inbred A are developed. These isolines are homozygotic for the same desirable alleles, but differ in that one isoline is homozygotic for the gene encoding the biotin-binding protein, while the other isoline lacks this gene. An isoline may be developed by first transgenically introducing a gene encoding a biotin-binding protein into an inbred line, then selfing the transgenic line to obtain the homozygote expressing the biotin-binding protein, as described above.

The present invention, thus generally described, will be understood more readily by reference to the following examples, which are provided by way of illustration and are not intended to be limiting of the present invention.

### EXAMPLE 1

### Preparation of Male-Sterile Transgenic Plants by High Expression of a MCCase Gene Fragment Encoding a Biotin-Binding Polypeptide

Expression vectors comprising a gene encoding a biotin-binding protein are used to transform plant material to make transgenic plants that express the biotin-binding polypeptide. In this example, the transformed plant material is maize cells in tissue culture, which are regenerated to whole, male-sterile maize plants.

The expression vector (“MCCase expression construct”) carries a gene encoding a fragment of the biotin-binding subunit of soybean 3-methylcrotonoyl-CoA carboxylase MCCase). Song et al., Proc. Natl. Acad. Sci. 91: 5779-5783. The biotin-binding domain of the MCCase biotin subunit is centered around the conserved biotin-binding motif AMKM, located at amino acid residues of 693-696 of the subunit, and includes a valine residue 33 amino acids downstream from the lysine residue that is covalently modified with biotin. A hydrophobic residue at this position is conserved among similar biotin-binding proteins and is believed responsible for the specificity of biotinylation of the lysine residue. For this example, the MCCase polypeptide contains amino acid residues 560-731, using the numbering scheme of Song et al. Id.

The gene is under control of the MS*5126 promoter, and it contains a PINII terminator sequence. pPHI610, an expression vector carrying the bar gene under control of the double 35S promoter, and also carrying a PINII terminator sequence, is co-transformed with the expression construct, allowing the selection of transgenic plants by treatment with bialophos. pPHI610 is described in U.S. Pat. No. 5,962,769. The expression vectors are co-transformed into embryogenic suspension cultures derived from type II embryogenic culture, according to the method of Green et al., MOLECULAR GENETICS OF PLANTS AND ANIMALS, Downey et al., eds., Academic Press, NY, 20, 147 (1983). The cultures are maintained in Murashige and Skoog (“MS”) medium as described in Murashige et al., Physio. Plant 15: 453 (1962) supplemented with 2,4-dichlorophenoxyacetic acid (2,4-D) at 2 mg/L and sucrose at 30 g/L. The suspension cultures are passed through a 710 μm sieve 7 days prior to the experiment and the filtrate is maintained in MS medium.

Cells are harvested from the suspension culture by vacuum filtration on a Buchner funnel (Whatman No. 614), and 100 ml (fresh weight) of cells are placed in a 3.3 cm petri plate. The cells are dispersed in 0.5 mL fresh culture medium to form a thin layer of cells. The uncovered petri plate is placed in the sample chamber of a particle gun device manufactured by Biolistics Inc. (Geneva, N.Y.). A vacuum pump is used to reduce the pressure in the chamber to 0.1 atmosphere to reduce deceleration of the microparticles by air friction. The cells are bombarded with tungsten particles having an average diameter of about 1.2 μm (GTE Sylvania Precision Materials Group, Towanda, Pa.). An equal mixture of the MCCase expression construct and pPHI610 are loaded onto the microparticles by adding 5 μl of a DNA solution (1 μg of DNA per 100 μl) in TE buffer at pH 7.7 to 25 μl of a suspension of 50 mg of tungsten particles per ml distilled water in a 1.5 ml Eppendorf tube. The particles aggregate and settle.

Cultures of transformed plant cells containing the foreign genes are cultivated for 4-8 weeks in 560R medium (N6-based medium with 1 mg/ml bialaphos). This medium selects for cells that express the bar gene.

Embryo formation is then induced in the embryogenic cultures and the cells germinate into plants. A two-culture medium sequence is used to germinate somatic embryos observed on callus maintenance medium. The callus is transferred first to a culture medium (maturation medium) containing 5.0 mg/L indoleacetic acid (IAA) for 10 to 14 days while callus proliferation continues. Callus loading is at 50 mg per plate to optimize recovery per unit mass of material.

The callus is then transferred from maturation medium to a second culture medium containing a reduced level of IAA (1 mg/L). Cultures then are placed in the light. Germinating somatic embryos are characterized by a green shoot elongating with a connecting root access. Somatic embryos then are transferred to medium in a culture tube (150×25 mm) for an additional 10-14 days. At this time, the plants are about 7-10 cm tall, and are of sufficient size and vigor to be “hardened” to greenhouse conditions.

To harden regenerated plants, plants are removed from the sterile containers and solidified agar medium is rinsed off the roots. The plantlets are placed in a commercial potting mix in a growth chamber with a misting device to maintain relative humidity near 100%. After 3-4 weeks in the misting chamber, the plants are robust enough for transplantation to the field.

Plants in the field are analyzed by observing male sterility. Selected plants are then further analyzed for presence of the MCCase gene fragment by PCR, essentially according to the procedure of Song et al., id. Biotin-binding activity in tissue extracts is determined by Western blotting with ¹²⁵I-streptavidin, also according to Song et al. Plants chosen for further analysis are male-sterile and express the MCCase biotin-binding polypeptide at high levels in anther tissue, because the expression of the MCCase gene fragment is driven by the anther-selective Ms*5126 promoter.

### EXAMPLE 2

### Use of Agrobacterium Strains Containing a Binary Vector Including a DNA Sequence Encoding the MCCase Biotin-Binding Polypeptide to Generate Male-Sterile Transgenic Soybean Plants

A method for forming transgenic soybean plants is that described in U.S. patent application Ser. No. 07/920,409, now abandoned, which is hereby incorporated by reference. Soybean seed (GLYCINE MAX), of Pioneer variety 9341, is surface sterilized by exposure to chlorine gas evolved in a glass bell jar. Gas is produced by adding 3.5 ml hydrochloric acid (34 to 37% w/w) to 100 ml sodium hypochlorite (5.25 % w/w). Exposure is for 16 to 20 hours in a container approximately 1 cubic ft in volume. Surface sterilized seed is stored in petri dishes at room temperature. Seed is germinated by plating an 1/10 strength agar solidified medium according to Gambourg (B5 basal medium with minimal organics, Sigma Chemical Catalog No. G5893, 0.32 gm/L sucrose; 0.2% weight/volume 2-(N-morpholino) ethanesulfonic acid (MES) (3.0 mM), without plant growth regulators and culturing at 28° with a 16-hour day length and cool white fluorescent illumination of approximately 20 μEm⁻² S1. After 3 or 4 days, seed is prepared for co-cultivation. The seed coat is removed and the elongating radical is removed 3 to 4 mm below the cotyledons.

Overnight cultures of Agrobacterium tumefaciens strain LBA4404 harboring the MCCase expression construct, which has been designed for expression in soybeans, are grown to log phase in minimal A medium containing tetracycline, 1 μg/ml, and are pooled and an optical density measurement at 550 nm is taken. Sufficient volume of the culture is placed in 15 mL/conical centrifuge tubes, such that upon sedimentation between 1 and 2×10¹⁰ cells are collected in each tube with 10⁹ cells/ml. Sedimentation is by centrifugation at 6,000× g for 10 min. After centrifugation, the supernatant is decanted and the tubes are held at room temperature until the inoculum is needed, but not longer than 1 hour.

Inoculations are conducted in batches such that each plate of seed is treated with a newly resuspended pellet of Agrobacterium. Bacterial pellets are resuspended individually in 20 ml inoculation medium, containing B5 salts (G5893), 3.2 g/L; sucrose, 2.0% w/v; 6-benzylaminopurine (BAP), 45 μm; indolebutyric-acid (IBA), 0.5 μM; acetosyringone (AS), 100 μM; buffered to pH 5.5 with MES, 10 mM. Resuspension is achieved by vortexing. The inoculum is then poured into a petri dish containing prepared seed and the cotyledonary nodes are macerated with a surgical blade. This is accomplished by dividing seed in half by longitudinal section through the shoot apex, preserving the two whole cotyledons. The two halves of the shoot apex are then broken off their respective cotyledons by prying them away with a surgical blade. The cotyledonary node is then macerated with a surgical blade by repeated scoring along the axis of symmetry. Care is taken not to cut entirely through the explant to the axial side. Explants are prepared in roughly five minutes and then incubated for 30 minutes at room temperature with bacteria but without agitation. After 30 minutes, the explants are transferred into plates of the same medium solidified with GELRITE (Merck & Company Inc.), 0.2% w/v. Explants are embedded with adaxial side up and leveled with the surface of the medium and cultured at 22° C. for 3 days under cool white fluorescent light, approximately 20 μEm⁻² s⁻¹.

After 3 days, the explants are moved to liquid counter-selection medium containing B5 salts (G5893), 3.2 g/l; sucrose, 2% w/v; BAP, 5 μM; IBA, 0.5 μM; vancomycin, 200 μg/ml; cefotaxime, 500 μg/ml, buffered to pH 5.7 with MES, 3 mM. Explants are washed in each petri dish with constant slow gyratory agitation at room temperature for four days. Counter-selection medium is replaced four times.

The explants are then picked to agarose-solidified selection medium containing B5 salts (G5893), 3.2 g/l; sucrose, 2% w/v; BAP, 5.0 μM; IBA, 0.5 μM; kanamycin sulfate, 50 μg/ml; vancomycin, 100 μg/ml; cefotaxime, 30 μg/ml; timentin, 30 μg/ml, buffered to pH 5.7 with MES, 3 mM. Selection medium is solidified with SEAKEM AGAROSE, 0.3 % w/v. The explants are embedded in the medium, adaxial side down and cultured at 28° with a 16 hour day length in cool white fluorescent illumination of 60 to 80 μEm⁻² s⁻¹.

After two weeks explants are again washed with liquid medium on the gyratory shaker. The wash is conducted overnight in counter-selection medium containing kanamycin sulfate, 50 μg/ml. The following day, explants are picked to agarose/solidified selection medium. They are embedded in the medium adaxial side down and cultured for another two week period.

After one month on selection medium, transformed tissue is visible as green sectors of regenerating tissue against a background of bleached non-healthy tissue. Explants without green sectors are discarded and explants with green sectors are transferred to elongation medium containing B5 salts (G5893), 3.2 g/l; sucrose, 2% w/v; IBA, 3.3 μM; gibberellic acid, 1.7 μM; vancomycin, 100 μg/ml; cefotaxime, 30 μg/ml; and timentin, 30 μg/ml, buffered to pH 5.7 with MES, 3 mM. Elongation medium is solidified with GELRITE, 0.2% w/v. The green sectors are embedded adaxial side up and cultured as before. Culture is continued on this medium with transfers to fresh plates every two weeks. When shoots are 0.5 cm in length they are excised at the base and placed in rooting medium in 13×100 ml test tubes. Rooting medium consists of B5 salts (G5893), 3.2 g/l; sucrose, 15 g/l; nicotinic acid, 20 μM; pyroglutamic acid (PGA), 900 mg/L and IBA, 10 μM. The rooting medium is buffered to pH 5.7 with MES, 3 mM and solidified with GELRITE at 0.2% w/v. After 10 days, the shoots are transferred to the same medium without IBA or PGA. Shoots are rooted and held in these tubes under the same environmental conditions as before.

When a root system is well established the plantlet is transferred to sterile soil. Temperature, photoperiod and light intensity remain the same as before.

The presence of the MCCase gene fragment and the expression of the MCCase biotin-binding polypeptide in transgenic soybean plants is confirmed and quantitated as described above. Stability of expression can be evaluated by these same methods over successive generations. Male sterility is correlated with expression of the MCCase gene fragment in the soybeans.

### EXAMPLE 3

### Preparation of Male-Sterile Sunflower Plants by Expression of the MCCase Gene Fragment

An expression cassette encoding the MCCase biotin-binding polypeptide is used to generate transgenic sunflower plants and seeds. The MCCase gene fragment is inserted into an expression cassette under control of the ubiquitin promoter. This expression cassette is then subcloned into a binary vector such as pPHI5765 using the EcoR1 site. The binary vector is then transferred into an Agrobacterium tumefaciens helper strain.

Sunflower plants are transformed with Agrobacterium strain LBA4404 after microparticle bombardment as described by Bidney et al., Plant Mol. Biol. 18: 301 (1992). Briefly, seeds of Pioneer Sunflower Line SMF-3 are dehulled and surface sterilized. The seeds are imbibed in the dark at 26° C. for 18 hours on filter paper moistened with water. The cotyledons and root radical are removed and meristem explants cultured on 374BGA medium (MS salts, Shephard vitamins, 40 mg/L adenine sulfate, 3% sucrose, 0.8% phytagar pH 5.6 plus 0.5 mg/L of BAP, 0.25 mg/L, IAA and 0.1 mg/L GA). Twenty-four hours later, the primary leaves are removed to expose the apical meristem and the explants are placed with the apical dome facing upward in a 2 cm circle in the center of a 60 mm by 20 mm petri plate containing water agar. The explants are bombarded twice with tungsten particles suspended in TE buffer or with particles associated with an expression plasmid containing the avidin gene. The meristem explants are co-cultured on 374BGA medium in the light at 26° C. for an additional 72 hours of co-culture.

Agrobacterium treated meristems are transferred following the 72 hour co-culture period to medium 374 (374BGA with 1 % sucrose and no BAP, IAA or GA₃) and supplemented with 250 μg/ml cefotaxime. The plantlets are allowed to develop for an additional two weeks under 16 hour day and 26° C. incubation conditions to green or bleach. Plantlets are transferred to medium containing kanamycin and allowed to grow. The presence of avidin in the plants is confirmed and quantitated as described in Example 2. The presence of male sterility is found to correlate with expression of the MCCase biotin-binding polypeptide by the plants.

Although the foregoing refers to particular preferred embodiments, it will be understood that the present invention is not so limited. Various modifications to these embodiments are within the skill of the artisan. All publications and patent applications mentioned in this specification are herein incorporated by reference in their entirety.

What is claimed is:

1. A method of producing a male-sterile plant, comprising
(a) transforming a plant cell with an expression vector that comprises a plant-compatible promoter operably linked to a nucleotide sequence encoding a biotin-binding polypeptide, and
(b) regenerating a transgenic plant from the transformed cell, whereby expression of the biotin-binding polypeptide causes male sterility of the transformed plant,
wherein the nucleotide sequence encodes a biotin-binding polypeptide selected from the group consisting of SBP65, acetyl coenzyme A carboxylase, methylcronotyl-coenzyme A carboxylase, carbon-dioxide ligase, and pyruvate decarboxylase.

2. The method of claim 1, wherein the biotin-binding polypeptide is a subunit or biotin-binding fragment of SBP65, acetyl coenzyme A carboxylase, methylcronotyl-coenzyme A carboxylase, carbon-dioxide ligase, or pyruvate decarboxylase.

3. The method of claim 2, wherein the biotin-binding polypeptide fragment is at least 30 contiguous amino acids in length.

4. The method of claim 2, wherein the biotin-binding polypeptide fragment is at least 50 contiguous amino acids in length.

5. The method of claim 2, wherein the biotin-binding polypeptide fragment is at least 100 contiguous amino acids in length.

6. The method of claim 1, wherein the biotin-binding protein is a variant of a naturally occurring biotin-binding polypeptide, wherein the variant possesses biotin-binding activity.

7. The method of claim 6, wherein the variant differs from the naturally occurring sequence of the biotin-binding polypeptide by no more than 50 amino acid substitutions, insertions, or deletions.

8. The method of claim 7, wherein the variant differs from the naturally occurring sequence of the biotin-binding polypeptide by no more than 100 amino acid substitutions, insertions, or deletions.

9. The method of claim 1, wherein the nucleotide sequence encoding a biotin-binding polypeptide is operably linked to a nucleotide sequence encoding a signal sequence.

10. The method of claim 9, wherein the signal sequence is a chloroplast-targeting signal sequence.

11. The method of claim 1, wherein the plant compatible promoter is selectively expressed in a tissue that is critical for pollen formation or function.

12. The method of claim 11, wherein the tissue is anther tissue.

13. The method of claim 12, wherein the promoter is selected from the group consisting of Ms*5126, SGB6, and G9.

14. A male-sterile plant comprising an expression vector that comprises a plant-compatible promoter operably linked to a nucleotide sequence encoding a biotin-binding polypeptide, wherein the nucleotide sequence encodes a biotin-binding polypeptide selected from the group consisting of SBP65, acetyl coenzyme A carboxylase, methylcronotyl-coenzyme A carboxylase, carbon-dioxide ligase, and pyruvate decarboxylase.

15. The plant of claim 14, wherein the nucleotide sequence encodes a biotin-binding fragment or subunit of a polypeptide selected from the group consisting of SBP65, an acetyl coenzyme A carboxylase, a methylcronotyl-coenzyme A carboxylase, a carbon-dioxide ligase, and a pyruvate decarboxylase.

16. The plant of claim 14, wherein the biotin-binding polypeptide fragment is at least 30 contiguous amino acids in length.

17. The plant of claim 16, wherein the biotin-binding polypeptide fragment is at least 50 contiguous amino acids in length.

18. The plant of claim 17, wherein the biotin-binding polypeptide fragment is at least 100 contiguous amino acids in length.

19. The plant of claim 14, wherein the nucleotide sequence encoding a biotin-binding polypeptide is operably linked to a nucleotide sequence encoding a signal sequence.

20. The plant of claim 19, wherein the signal sequence is a chloroplast-targeting signal sequence.

21. The plant of claim 14, wherein the biotin-binding protein is a variant of a naturally occurring biotin-binding polypeptide, wherein the variant possesses biotin-binding activity.

22. The plant of claim 21, wherein the variant differs from the naturally occurring sequence of the biotin-binding polypeptide by no more than 50 amino acid substitutions, insertions, or deletions.

23. The plant of claim 22, wherein the variant differs from the naturally occurring sequence of the biotin-binding polypeptide by no more than 100 amino acid substitutions, insertions, or deletions.

24. A method of producing a male-fertile hybrid plant, comprising the steps of:
(a) producing a first male-sterile parent plant comprising an expression vector comprising a plant compatible promoter operably linked to nucleotide sequence encoding a biotin-binding polypeptide, wherein the expression of the biotin-binding polypeptide causes male sterility, and wherein the nucleotide sequence encodes a biotin-binding polypeptide selected from the group consisting of SBP65, acetyl coenzyme A carboxylase, methylcronotyl-coenzyme A carboxylase, carbon-dioxide ligase, and pyruvate decarboxylase;
(b) producing a second transgenic parent plant expressing a second foreign gene; and
(c) cross-fertilizing the first parent with the second parent to produce a hybrid plant,
wherein the hybrid plant expresses the second foreign gene, and wherein the product of the second foreign gene reduces expression or function of the biotin-binding polypeptide in the hybrid plant, thereby producing a male-fertile hybrid plant.

25. The method of claim 24, wherein the plant compatible promoter is selectively expressed in a tissue that is critical for pollen formation or function.

26. The method of claim 24, wherein the product of the second foreign gene is selected from the group consisting of an antisense molecule, a ribozyme, an external guide sequence, and an antibody against the biotin-binding protein.

27. The method of claim 24, wherein the activity of the plant compatible promoter is regulated by an operon, and wherein the product of the second foreign gene is a repressor protein that is capable of binding the operon and repressing the activity of the plant compatible promoter.

28. A method of propagating a male-sterile plant, comprising the steps of:
(a) producing a male-sterile plant comprising an expression vector comprising a plant compatible promoter operably linked to nucleotide sequence encoding a biotin-binding polypeptide, wherein the expression of the biotin-binding polypeptide causes male sterility, and wherein the nucleotide sequence encodes a biotin-binding polypeptide selected from the group consisting of SBP65, acetyl coenzyme A carboxylase, methylcronotyl-coenzyme A carboxylase, carbon-dioxide ligase, and pyruvate decarboxylase;
(b) spraying the male-sterile plant with a solution comprising biotin in an amount sufficient to restore pollen production;
(c) selfing the male-sterile plant in which pollen production has been restored; and
(d) collecting the seeds produced by the plant.

29. The method of claim 28, wherein the plant compatible promoter is selectively expressed in a tissue that is critical for pollen formation or function.

30. A method of propagating a male-sterile plant, comprising the steps of:
(a) producing a male-sterile plant comprising an expression vector comprising
(i) a first plant compatible promoter operably linked to a first nucleotide sequence encoding a biotin-binding polypeptide, wherein the expression of the biotin-binding polypeptide causes male sterility, and wherein the nucleotide sequence encodes a biotin-binding polypeptide selected from the group consisting of SBP65, acetyl coenzyme A carboxylase, methylcronotyl-coenzyme A carboxylase, carbon-dioxide ligase, and pyruvate decarboxylase; and
(ii) a second inducible promoter operably linked to a second nucleotide sequence encoding a gene product that reduces expression of the biotin-binding polypeptide;
(b) spraying the male-sterile plant with a solution comprising an inducer in an amount sufficient to cause expression of the second gene at a level sufficient to reduce the expression of the first gene, thereby restoring pollen production;
(c) self-pollinating the male-sterile plant in which pollen production has been restored; and
(d) collecting the seeds produced by the plant.

31. The method of claim 30, wherein the plant compatible promoter is selectively expressed in a tissue that is critical for pollen formation or function.

32. The method of claim 30, wherein the product of the second foreign gene is an antisense molecule, a ribozyme gene, an external guide sequence, or an antibody against the biotin-binding protein.

33. A method for producing hybrid seeds, comprising the steps of:
(a) producing a first male-sterile parent plant comprising an isolated DNA molecule comprising a plant compatible promoter operably linked to nucleotide sequence encoding a biotin-binding polypeptide, wherein the expression of the biotin-binding polypeptide causes male sterility, and wherein the nucleotide sequence encodes a biotin-binding polypeptide selected from the group consisting of SBP65, acetyl coenzyme A carboxylase, methylcronotyl-coenzyme A carboxylase, carbon-dioxide ligase, and pyruvate decarboxylase;
(c) cross-fertilizing the first parent plant with a second parent plant to produce hybrid seeds; and
(d) harvesting the hybrid seeds from the first parent plant.

34. The method of claim 33, wherein the plant compatible promoter is selectively expressed in a tissue that is critical for pollen formation or function.

35. The method of claim 33, wherein the first plant is homozygotic for the nucleotide sequence encoding a biotin-binding polypeptide.

36. The method of claim 33, wherein the first plant is hemizygotic for the nucleotide sequence encoding a biotin-binding polypeptide.

37. The method of claim 33, wherein the second parent plant carries one or more genes controlling a desired grain trait, and wherein the hybrid seeds carry the one or more genes controlling the desired gene trait.

38. The method of claim 37, wherein the desired grain trait is selected from the group consisting of oil, protein, and starch content.

39. A method for producing F1 hybrid seeds, comprising the steps of:
(a) producing a first inbred parent plant which is male-sterile and comprises a nucleotide sequence encoding a biotin-binding polypeptide operably linked to a plant-compatible promoter sequence, wherein the first parent plant is homozygotic for the nucleotide sequence encoding a biotin-binding polypeptide, and wherein the nucleotide sequence encodes a biotin-binding polypeptide;
(b) producing a second inbred parent plant which is male-fertile;
(c) cross-fertilizing the first parent with the second parent to produce a third male-sterile parent plant which is hemizygotic for the nucleotide sequence encoding a biotin-binding polypeptide;
(d) producing a fourth parent plant which is male-fertile;
(e) cross-fertilizing the third parent plant with the fourth parent plant to produce hybrid seeds; and
(f) harvesting the hybrid seeds from the third parent plant.

40. The method of claim 39, wherein the plant compatible promoter is selectively expressed in a tissue that is critical for pollen formation or function.

41. The method of claim 39, wherein the first and second parent plants are homozygotic for one or more genes controlling a second desired gene trait.

42. The method of claim 39, wherein the biotin-binding polypeptide is selected from the group consisting of SBP65, acetyl coenzyme A carboxylase, methylcronotyl-coenzyme A carboxylase, carbon-dioxide ligase, and pyruvate decarboxylase.

43. The method of claim 39, wherein the fourth parent plant carries one or more genes controlling a desirable trait.

44. The method of claim 39, wherein the desired grain trait is selected from the group consisting of oil, protein, and starch content.
